(19) United States
(12) Patent Application Publication
(10) Pub. No.: US 2009/0148894 A1
(43) Pub. Date: Jun. 11, 2009

(54) METHODS FOR OPTIMIZING THE SECRETION OF PROTEIN IN PROKARYOTES

(76) Inventors: Sheldon E. BROEDEL, Baltimore, MD (US); Sharon M. PAPCIAK, Baltimore, MD (US); Joel WEINER, Edmonton (CA)

Correspondence Address: EDWARD YOO C/O BENNETT JONES, 1000 ATCO CENTRE, 10035 - 105 STREET, EDMONTON, ALBERTA, AB, T5J3T2, CA

(21) Appl. No.: 12/045,364

(22) Filed: Mar. 10, 2008

Related U.S. Application Data
(63) Continuation-in-part of application No. 11/203,168, filed on Aug. 15, 2005.
(60) Provisional application No. 60/905,486, filed on Mar. 8, 2007.

(51) Int. Cl. C12P 21/00; C12N 15/63; C12N 1/00; C12N 1/21

(52) U.S. Cl. 435/69.1; 435/320.1; 435/243; 435/252.33

(57) ABSTRACT

Methods are provided for producing recombinant proteins by utilizing expression vectors carrying nucleic acids encoding the proteins, and secretory signal sequences to direct the secretion of the proteins to the periplasm or extracellular medium. Expression vectors which encode a fusion protein comprising a carrier protein and the protein are also provided, as are host cells transformed with the expression vectors.

### CROSS REFERENCE TO RELATED APPLICATIONS

This application claims priority to U.S. Provisional Application No. 60/905,486, filed Mar. 8, 2007, and also claims priority to U.S. patent application Ser. No. 11/203,168 filed on Aug. 15, 2003, the entire contents of both which are hereby incorporated herein by reference in their entirety.

### FIELD OF THE INVENTION

The present invention relates to the field of recombinant protein production in prokaryotes such as Escherichia coli.

### BACKGROUND OF THE INVENTION

Prokaryotes have been widely used for the production of recombinant proteins. Controlled expression of the desired polypeptide or protein is accomplished by coupling the gene encoding the protein through recombinant DNA techniques behind a promoter, the activity of which can be regulated by external factors. This expression construct is carried on a vector, most often a plasmid. Introduction of the plasmid carrying the expression construct into a host bacterium and culturing that organism in the presence of compounds which activate the promoter results in high levels of expression of the desired protein. In this way, large quantities of the desired protein can be produced.

E. coli is the most commonly used prokaryote for protein production. Many different varieties of plasmid vectors have been developed for use in E. coli to build expression vectors. The different variations employ several different types of promoters, selectable markers, and origins of replication where each of the different configurations imparts a unique property to origins of replication where each of the different configurations imparts a unique property to the expression vector. In the most common arrangement, the expressed protein accumulates in the cytoplasm. While this approach is useful for some proteins, not all proteins can be accumulated in the cytoplasm in an active state. Often, when the desired protein is produced at high levels relative to the host proteins, is toxic to the host cell, or has particular structural properties, the protein accumulates as an insoluble particle known as an inclusion body. Proteins which accumulate as inclusion bodies are difficult to recover in an active form.

One means of solving this problem is to export the desired protein to the periplasm between the inner and outer membranes (Choi et al., 2004; Cornelis, 2000). By placing a signal sequence in front of the coding sequence of the desired protein, the expressed protein can be directed to a particular export pathway (U.S. Pat. No. 5,047,334 to Petro et al., U.S. Pat. No. 4,963,495 to Chang et al.). Known export pathways in E. coli include the SecB-dependent (SEC) (Fekkes et al., 1999), the twin-arginine translocation (TAT) (Sargent et al., 2005; Fisher et al., 2004), and the signal recognition particle (SRP) pathway (Koch et al., 2003; Valent, 2001; Luirink et al., 2004). Translocation in the SEC or TAT pathway is via a post-translational mechanism, whereas the SRP pathway translocation is co-translational. Proteins translocated by the SEC pathway are unfolded prior to export and then refolded in the periplasm. In the TAT pathway, the proteins are translocated in a folded state.

The selected export pathway is encoded in the signal sequence placed in front of the coding sequence of the desired protein within an expression vector. Currently available expression vectors incorporate signal sequences derived from proteins whose export is directed through the SEC pathway, such that the proteins accumulate in the periplasm (Table 1 taken from Choi et al., 2004):

TABLE 1
Signal sequences used to secrete proteins in E. coli

| Signal Sequences | Protein |
|---|---|
| PelB | Pectate lyase B from Erwinia carotovora |
| OmpA | Outer-membrane protein A |
| StlI | Heat-stable enterotoxin 2 |
| Endo | Endoxylanase from Bacillus sp. |
| PhoA | Alkaline phosphatase |
| OmpF | Outer-membrane pore protein F |
| PhoE | Outer-membrane pore protein E |
| MalE | Maltose-binding protein |
| OmpC | Outer-membrane protein C |
| Lpp | Murein lipoprotein |
| LamB | λ receptor protein |
| OmpT | Protease VII |
| LTB | Heat-labile enterotoxin subunit B |

Although numerous proteins have been successfully produced by this method, many proteins are not exported correctly or in a functional state due to aggregation in the cytoplasm; lysis of the cells; incorrect folding; limitations to translocation or proteolytic degradation (Jung et al., 1997; Krebber et al., 1996; Brinkmann et al., 1995; Rodi et al., 2002; Wulfing et al., 1993). The efficiency of translocation of a given protein depends on the signal sequence used and does not guarantee the secretion of a protein. Since the SEC and TAT pathways require the use of chaperone proteins (known to be substrate (protein)-specific; Baneyx et al., 2004) to effect translocation, many heterologous proteins when expressed in E. coli with SEC signal sequences cannot be exported due to lack of recognition by the host chaperones. For SEC-based translocation, the chaperones must retain the substrate protein in a partially unfolded state which is not likely possible with every protein. For secretion using the TAT pathway, some proteins can not be exported in a fully folded state due to steric interference. Protein export using the SRP pathway is likely to be hindered by the nature of the protein sequence; for example, where an amino acid sequence with a series of charged or hydrophobic residues might be blocked from being translocated due to strong protein-protein interactions. Not all proteins can be translocated equally well by any one export mechanism.

There is thus a need for signal sequences and expression vectors including such sequences to facilitate the selection of an appropriate export pathway which is most suitable for the production of a desired protein.

### SUMMARY OF THE INVENTION

The present invention relates to a method for producing a recombinant protein, polypeptide or peptide of interest through secretion of the recombinant protein, polypeptide or peptide to the periplasm or extracellular growth medium. The method utilizes expression vectors carrying particular secretory signal sequences to direct the secretion of the recombinant protein, polypeptide or peptide to the periplasm or extracellular growth medium via the SEC, TAT or SRP export pathways.

In one aspect, the invention provides an expression vector capable of directing the expression and secretion of a protein, polypeptide or peptide in a suitable host cell, wherein the expression vector comprises a nucleic acid encoding a fusion protein comprising YebF, or a biologically active variant or portion thereof, and the protein, polypeptide or peptide, operably linked to control sequences compatible with the host cell, and a secretory signal sequence for directing the secretion of the fusion protein.

In one embodiment, the expression vector comprises a signal sequence comprising one of SEQ ID NO: 4, SEQ ID NO: 5, SEQ ID NO: 6, SEQ ID NO: 7, SEQ ID NO: 8 or SEQ ID NO: 9. In one embodiment, the expression vector is comprised of plasmid pAES30, pAES31, pAES32, pAES33, pAES34, or pAES35. In one embodiment, the expression vector is plasmid pAES40. In one embodiment, the expression vector is for use in a prokaryotic host cell, for example, Escherichia coli or a strain thereof.

In another aspect, the invention provides an isolated host cell transformed by any of the above expression vectors, so that the cell expresses and secretes a protein, polypeptide or peptide encoded by the nucleic acid. In one embodiment, the host cell is a prokaryotic host cell, for example, Escherichia coli or a strain thereof.

In yet another aspect, the invention provides a method of optimized production of a protein, polypeptide or peptide comprising the steps of:
(a) choosing an expression vector as described herein, comprising a signal sequence associated with one of SEC, TAT, or SRP export pathway, wherein said choice is made having regard to known information about the protein, polypeptide or peptide, or experimental information from expression studies of the signal sequence and the protein, polypeptide or peptide;
(b) transforming a suitable host cell with the chosen expression vector;
(c) culturing the transformed host cell under conditions conducive to the expression of the protein, polypeptide or peptide to generate a secreted protein, polypeptide or peptide; and
(d) recovering the secreted protein, polypeptide, or peptide from the host cell, from the culture medium comprising the host cell, or from an extract obtained from the host cell.

In one embodiment, the expression vector is selected from the group consisting of plasmids pAES30, pAES31, pAES32, pAES33, pAES34, pAES35 or pAES40.

### BRIEF DESCRIPTION OF THE DRAWINGS

The invention will now be described by way of an exemplary embodiment with reference to the accompanying simplified, diagrammatic, not-to-scale drawings.

FIGS. 1A, 1B and 1C depict the plasmid map of the expression vector pAES25.

FIG. 2 depicts the nucleotide sequence of the promoter, translation start site and multiple cloning site of plasmid pAES25.

FIGS. 3A and 3B depict the plasmid map and signal sequences of the expression vectors pAES30-35.

FIGS. 4A and 4B depict the plasmid map of the expression vector pAES40.

FIG. 5 is a graph showing luciferase activity in E. coli strains harboring signal sequence-SA-Luc constructs after induction.

FIG. 6 depicts two immunoblots showing the accumulation of YebF-Amy (left panel) and YebF-PhoA (right panel) in the growth medium and intracellularly.

FIG. 7 depicts the expression of YebF using different signal sequences.

FIG. 8 depicts the plasmid map of the expression vector pYebF-Amy2.

### DETAILED DESCRIPTION OF THE PREFERRED EMBODIMENTS

As will be apparent to those skilled in the art, various modifications, adaptations and variations of the foregoing specific disclosure can be made without departing from the scope of the invention claimed herein. The various features and elements of the described invention may be combined in a manner different from the combinations described or claimed herein, without departing from the scope of the invention.

The present invention relates to a method for producing a recombinant protein, polypeptide or peptide of interest through secretion of the recombinant protein, polypeptide or peptide to the periplasm or extracellular growth medium. The method utilizes expression vectors carrying particular secretory signal sequences to direct the secretion of the recombinant protein, polypeptide or peptide to the periplasm or extracellular growth medium via the SEC, TAT or SRP export pathways. The expression vectors facilitate the selection of the appropriate signal sequence and export pathway which are most suited for the protein, polypeptide or peptide to achieve successful secretion.

To facilitate understanding of the invention, the following definitions are provided.

“Expression” refers to transcription or translation, or both, as context requires.

An “expression vector” refers to a recombinant DNA molecule containing the appropriate control nucleotide sequences (e.g., promoters, enhancers, repressors, operator sequences and ribosome binding sites) necessary for the expression of an operably linked nucleotide sequence in a particular host cell. By “operably linked/linking” or “in operable combination” is meant that the nucleotide sequence is positioned relative to the control nucleotide sequences to initiate, regulate or otherwise direct transcription and/or the synthesis of the desired protein molecule. The expression vector may be self-replicating, such as a plasmid, and may therefore carry a replication site, or it may be a vector that integrates into a host chromosome either randomly or at a targeted site. The expression vector may contain a selection gene as a selectable marker for providing phenotypic selection in transformed cells. The expression vector may also contain sequences that are useful for the control of translation.

A “fusion” protein is a recombinant protein comprising regions derived from at least two different proteins. The term “fusion protein” as used herein refers to a protein molecule in which a protein, polypeptide or peptide of interest is fused to: YebF, a biologically active variant of YebF, or a biologically active portion of YebF (herein a “YebF, or a biologically active variant or portion thereof”). “Fused”, in one context means that nucleic acid encoding YebF, or a biologically active variant or portion thereof, is joined in frame to the nucleic acid encoding the protein, polypeptide or peptide of interest, to provide for a single amino acid chain when transcription and translation occur. In another context, “fused” may also be a reference to the joining of a protein, polypeptide or peptide of interest to YebF, or a biologically active variant or portion thereof.

A “secreted fusion protein” is the part of the fusion protein that is secreted into the growth medium. As is apparent, a secreted fusion protein will likely lack the amino acids that comprise the leader sequence of YebF, specifically MKKRGA FLGLLLVSAC ASVF (included in SEQ ID NO:1).

A “nucleotide” refers to a ribonucleotide or a deoxyribonucleotide. “Nucleic acid” refers to a polymer of nucleotides and may be single- or double-stranded. “Polynucleotide” refers to a nucleic acid that is twelve or more nucleotides in length.

A “nucleotide sequence of interest” refers to any nucleotide sequence that encodes a “protein, polypeptide or peptide sequence of interest,” the production of which may be deemed desirable for any reason, by one of ordinary skill in the art. Such nucleotide sequences include, but are not limited to, coding sequences of structural genes, regulatory genes, antibody genes, enzyme genes, etc., or portions thereof. The nucleotide sequence of interest may comprise the coding sequence of a gene from one of many different organisms.

A nucleotide sequence “encodes” or “codes for” a protein if the nucleotide sequence can be translated to the amino acid sequence of the protein. The nucleotide sequence may or may not contain an actual translation start codon or termination codon.

A “protein, polypeptide or peptide sequence of interest” is encoded by the “nucleotide sequence of interest.” The protein, polypeptide or peptide may be a protein from any organism, including but not limited to, mammals, insects, micro-organisms such as bacteria and viruses. It may be any type of protein, including but not limited to, a structural protein, a regulatory protein, an antibody, an enzyme, an inhibitor, a transporter, a hormone, a hydrophilic or hydrophobic protein, a monomer or dimer, a therapeutically-relevant protein, an industrially-relevant protein, or portions thereof.

A “peptide” is polymer of four to 20 amino acids, a “polypeptide” is a polymer of 21 to 50 amino acids and a “protein” is a polymer of more than 50 amino acids.

A “portion” when used in reference to a protein refers to fragments of that protein. The fragments may range in size from four amino acid residues to the entire amino acid sequence of the protein, minus one amino acid.

“Purified” or “to purify” refers to the removal of undesired components from a sample. For example, to purify the secreted protein from growth medium, may mean to remove other components of the medium (i.e., proteins and other organic molecules), thereby increasing the percentage of the secreted protein.

The terms “modified”, “mutant” or “variant” are used interchangeably herein, and refer to: (a) a nucleotide sequence in which one or more nucleotides have been added or deleted, or substituted with different nucleotides or modified bases or to (b) a protein, peptide or polypeptide in which one or more amino acids have been added or deleted, or substituted with a different amino acid. A variant may be naturally occurring, or may be created experimentally by one of skill in the art. A variant may be a protein, peptide, polypeptide or polynucleotide that differs (i.e., an addition, deletion or substitution) in one or more amino acids or nucleotides from the parent sequence.

In this regard, it is well understood in the art that certain alterations inclusive of mutations, additions, deletions and substitutions can be made to a reference nucleic acid or protein, whereby the altered nucleic acid or protein retains a particular biological function or activity, or perhaps displays an altered but nevertheless useful activity. Some deletions, insertions and substitutions will not produce radical changes in the characteristics in a protein or nucleic acid. However, while it may be difficult to predict the exact effect of the substitution, deletion or insertion in advance of doing so, one skilled in the art will appreciate that the effect can be evaluated by routine screening assays. For example whether a variant has a secretory function can be determined by assaying for whether the variant, or a fusion protein comprising the variant, is secreted into the medium, by the methods disclosed herein. Modifications of protein properties such as redox or thermal stability, hydrophobicity, susceptibility to proteolytic degradation, or the tendency to aggregate with carriers or into multimers may be assayed by methods well known to one of skill in the art.

Variants may be created experimentally using random mutagenesis, oligonucleotide-mediated (or site-directed) mutagenesis, PCR mutagenesis and cassette mutagenesis. Oligonucleotide-mediated mutagenesis is well known in the art using vectors that are either derived from bacteriophage M13, or that contain a single-stranded phage origin of replication. Production of single-stranded template is described, for example, in Sambrook et al. (1989). Alternatively, the single-stranded template may be generated by denaturing double-stranded plasmid (or other DNA) using standard techniques. Alternatively, linker-scanning mutagenesis of DNA may be used to introduce clusters of point mutations throughout a sequence of interest that has been cloned into a plasmid vector (Ausubel et al., 1990). Region-specific mutagenesis and directed mutagenesis using PCR may also be employed to construct variants according to the invention. With regard to random mutagenesis, methods include incorporation of dNTP analogs and PCR-based random mutagenesis.

“Periplasm” refers to a gel-like region between the outer surface of the cytoplasmic membrane and the inner surface of the lipopolysaccharide layer of gram-negative bacteria.

“Secretion” refers to the excretion of the recombinant protein that is expressed in a bacterium to the periplasm or extracellular growth medium.

“YebF” is a reference to the protein having the amino acid sequence of SEQ ID NO:1. “Mature YebF” is a reference to the protein having the amino acid sequence of SEQ ID NO:2. “yebF” is a reference to a nucleic acid or nucleotide sequence having the sequence of SEQ ID NO:3.

Standard recombinant DNA and molecular cloning techniques used herein are well known in the art and are described by Sambrook, J., Fritsch, E. F. and Maniatis, T., Molecular Cloning: A Laboratory Manual, Second Edition, Cold Spring Harbor Laboratory Press, Cold Spring Harbor, N.Y. (1989); Silhavy, T. J., Bennan, M. L. and Enquist, L. W., Experiments with Gene Fusions, Cold Spring Harbor Laboratory Cold Press Spring Harbor, N.Y. (1984); and Ausubel, F. M. et al., Current Protocols in Molecular Biology, published by Greene Publishing Assoc. and Wiley-Interscience (1990).

The present invention utilizes expression vectors carrying particular secretory signal sequences and a nucleotide sequence encoding a target protein, polypeptide or peptide to direct the secretion of the recombinant protein, polypeptide or peptide to the periplasm or to extracellular growth medium via the SEC, TAT or SRP export pathways. The expression vectors facilitate the selection of the appropriate secretory signal sequence and export pathway which are most suited for the protein, polypeptide or peptide to achieve successful secretion.

The expression vectors of the present invention can be constructed using techniques well known in the art (Sambrook et al., 1989; Ausubel et al., 1990). Briefly, the nucleotide sequence encoding the target protein, polypeptide or peptide is placed in operable combination with control nucleotide sequences (e.g., promoters, enhancers, repressors, operator sequences and ribosome binding sites) to initiate, regulate or otherwise direct transcription and/or the synthesis of the desired protein, polypeptide or peptide. The control nucleotide sequences include, for example, initiation signals such as start (i.e., ATG); transcription termination or stop codons; promoters which may be constitutive (i.e., continuously active) or inducible; and enhancers which increase the efficiency of expression. Secretory signal sequences are included to achieve secretion of the encoded protein, polypeptide or peptide from the cytoplasm into the periplasm or extracellularly.

It will be appreciated by those skilled in the art that the promoter which regulates the transcription of the nucleotide sequence encoding the target protein, polypeptide or peptide may be modified to increase or decrease the transcription rates. Likewise, the plasmid copy number may be increased or decreased by modifying the origin of replication. Both of these modifications would be expected to yield higher or lower levels of expression and thus higher or lower levels of accumulated protein, polypeptide or peptide. It will be appreciated by those skilled in the art that the promoter and copy number variants can be matched with the appropriate secretory signal sequence to effect optimum protein production.

Marker genes are included to allow selection of host cells bearing the desired expression vector including, but not limited to, antibiotic (e.g., ampicillin and kanamycin) resistance genes, or reporter genes (e.g., luciferase) which catalyze the synthesis of a visible reaction product. Ancillary sequences enhancing protein purification may also be included in the expression vector.

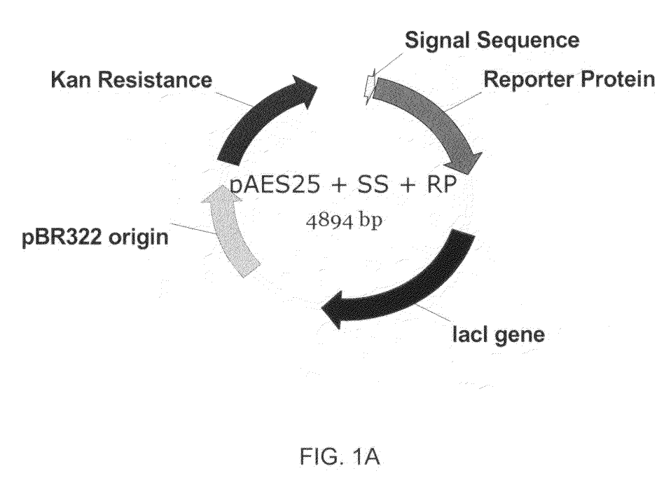

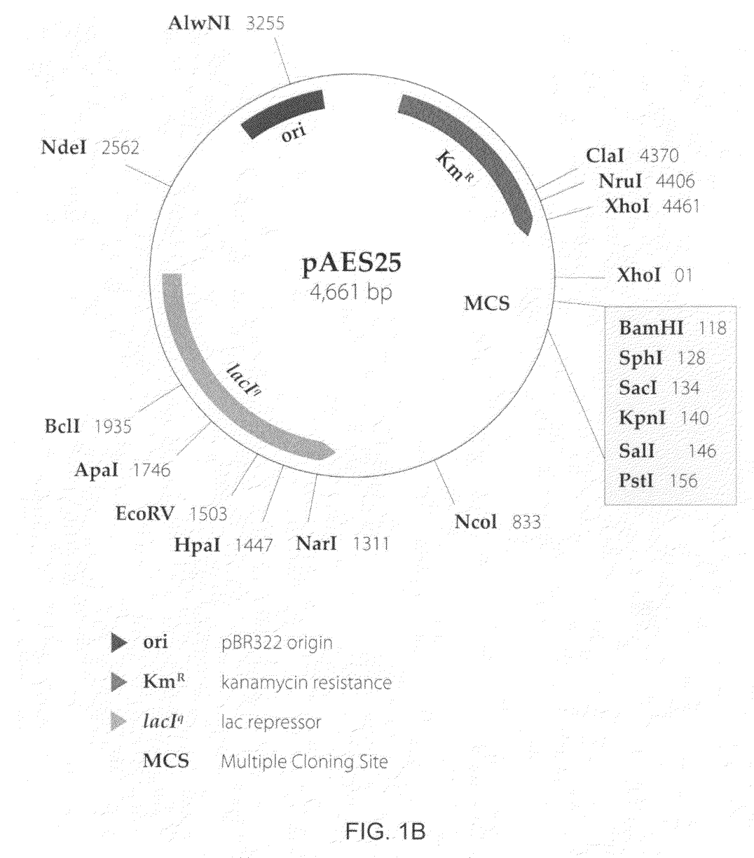

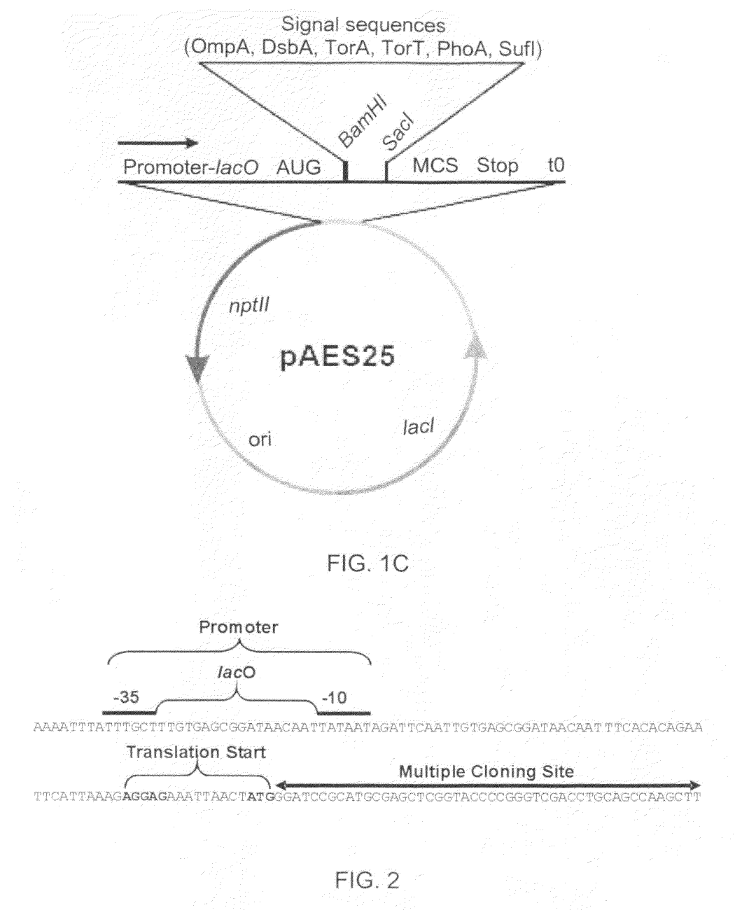

In one embodiment, the invention provides expression vectors comprising the basic plasmid map shown in FIGS. 1A, 1B and 1C. The base expression vector, pAES25 (SEQ ID NO:18) was constructed using standard techniques known in the art by coupling together the origin of replication (ori) from pBR322, a plasmid which is one of the most commonly used E. coli cloning vectors; the nptII gene (kanamycin-resistance); the lacI gene (lactose repressor); a promoter under the control of the lac operator/repressor system; a translation start site and a multiple cloning site (MCS). FIG. 2 depicts the nucleotide sequence from upstream of the promoter through the MCS. The promoter is located upstream of the translation start site which lies upstream of the MCS. The signal sequence is inserted into the MCS downstream of the promoter and translational start site. The coding sequence for the desired protein, polypeptide or peptide is inserted downstream of the signal sequence. A gene encoding an easily assayed reporter protein may be included to measure expression and protein secretion.

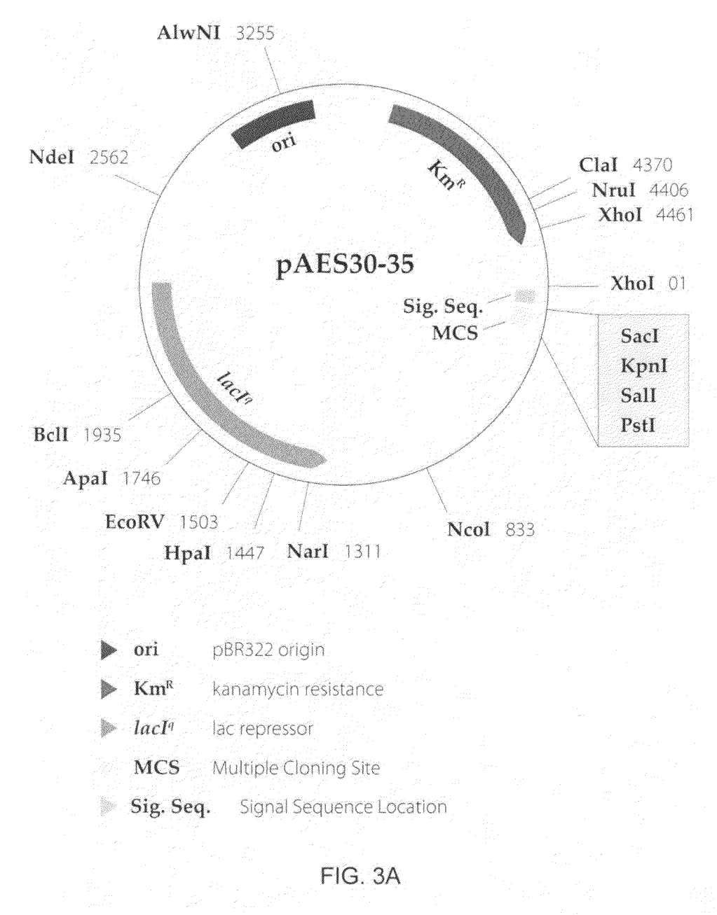

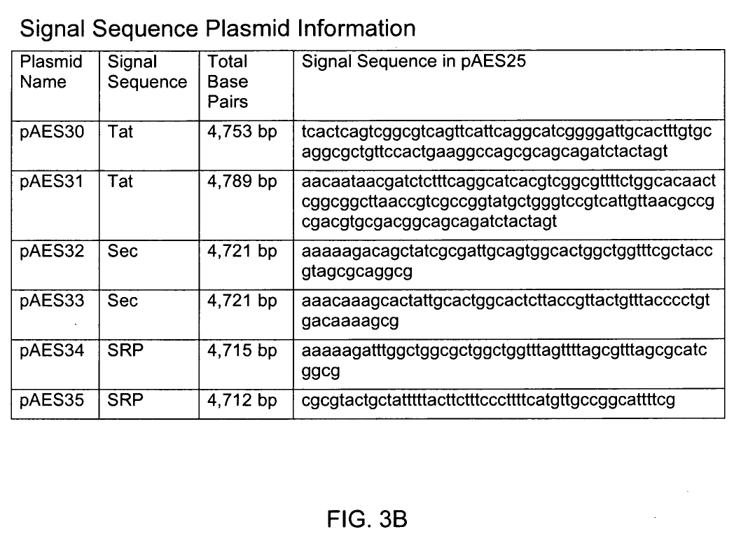

In one embodiment, the invention provides expression vectors comprising the plasmid map shown in FIGS. 3A and 3B. Each expression vector (pAES30-35) was constructed to carry a particular secretory signal sequence (Table 2), but is otherwise identical to pAES25. The signal sequences were selected to represent two signals for each of the SEC, TAT and SRP export pathways of E. coli (Table 3). FIG. 1C shows the plasmid map of pAES25 with the control region expanded out. DNA sequences encoding each of the signal sequences were synthesized chemically. The expression vectors were made by inserting the signal sequences into the MCS at the BamHI (5′ end) and SacI (3′ end) restriction sites of pAES25. The resulting plasmids retained the reading frame defined by the ATG start codon of pAES25 (FIG. 2).

TABLE 2
Signal sequences

| | |
|---|---|
| SEQ ID NO:4 | AAACAAAGCACTATTGCACTGGCACTCTTACCGTTACT |
| “PhoA” | GTTTACCCCTGTGACAAAAGCG |
| SEQ ID NO:5 | AAAAAGACAGCTATCGCGATTGCAGTGGCACTGGCTGG |
| “OmpA” | TTTCGCTACCGTAGCGCAGGCG |
| SEQ ID NO:6 | AAAAAGATTTGGCTGGCGCTGGCTGGTTTAGTTTTAGC |
| “DsbA” | GTTTAGCGCATCGGCG |
| SEQ ID NO:7 | CGCGTACTGCTATTTTTACTTCTTTCCCTTTTCATGTT |
| “TorT” | GCCGGCATTTTCG |
| SEQ ID NO:8 | TCACTCAGTCGGCGTCAGTTCATTCAGGCATCGGGGAT |
| “SufI” | TGCACTTTGTGCAGGCGCTGTTCCACTGAAGGCCAGCG |
| | CAGCAGATCTACTAGT |
| SEQ ID NO:9 | AACAATAACGATCTCTTTCAGGCATCACGTCGGCGTTT |
| “TorA” | TCTGGCACAACTCGGCGGCTTAACCGTCGCCGGTATGC |
| | TGGGTCCGTCATTGTTAACGCCGCGACGTGCGACGGCA |
| | GCAGATCTACTAGT |

TABLE 3
Signal sequences used to demonstrate differential secretion

| | | | Resulting Plasmid |
|---|---|---|---|
| Signal | | Amino Acid Sequence | (Alternate Plasmid |
| Sequence | Pathway | for Each Signal Sequence | Name) |
| PhoA | Sec | MKQSTIALALLPLLFTPVKTA (SEQ ID NO:10) | pAES25PhoASGL |
| | | | (pAES32) |
| OmpA | Sec | MKKTAIAIAVALAGFATVAQA (SEQ ID NO:11) | pAES25OmpASGL |
| | | | (pAES31) |
| DsbA | SRP | MKKIWLALAGLVLAFSASA (SEQ ID NO:12) | pAES25DsbASGL |
| | | | (pAES30) |
| TorT | SRP | MRVLLFLLSLFMLPAFS (SEQ ID NO:13) | pAES25TorTSGL |
| | | | (pAES35) |
| SufI | Tat | MSLSRRQFIQASGIALCAGAVPLKASA (SEQ ID NO:14) | pAES25SufISGL |
| | | | (pAES33) |
| TorA | Tat | MNNNDLFQASRRRFLAQLGGLTVAGMLGPSLLTPRRATA | pAES25TorASGL |
| | | (SEQ ID NO:15) | (pAES34) |

Knowledge about the primary or secondary structure of the target protein can be used to select the appropriate signal sequence and expression vector likely to achieve successful export of the protein. For example, if the target protein is known to have fast folding properties or once folded, is difficult to unfold, then the co-translational mechanism (SRP pathway) may be most suitable. It will be appreciated by those skilled in the art that, due to the degeneracy of the genetic code, modifications can be made to the nucleotide sequences encoding the signal sequences. Such modifications would alter the amino acid sequence such that the leader is more effective in delivering the target protein to the export machinery of the cell. In one embodiment, the modifications comprise replacement of specific amino acid residues of the sequences listed in Table 2 with other amino acid residues, or an increase or decrease in the length of the leader. It is expected that these sequence modifications could be carried on an expression vector similar to, but not necessarily identical, to pAES25. Further, modifications to the nucleotide sequence of genes encoding proteins involved in the export functions could increase their capacity to translocate proteins. Further, co-expression of these same proteins on expression vectors could increase the relative number of export machinery proteins and consequently could lead to higher export capacity.

In one embodiment, the invention provides the above expression vectors carrying signal sequences and a target gene to direct the secretion of the recombinant protein, polypeptide or peptide into the periplasm (as described in Example 1) or extracellular growth medium.

In one embodiment, the invention provides expression vectors carrying signal sequences, a target gene, and a carrier gene to direct the secretion of the recombinant protein, polypeptide or peptide into growth medium. In one embodiment, the invention comprises fusion of the target protein, polypeptide or peptide to the E. coli protein YebF, which is transported to the growth medium. YebF is a small (10.8 kDa) soluble endogenous protein which is naturally secreted into growth medium by E. coli cells. It effectively transports both small and large prokaryotic and eukaryotic proteins to the extracellular medium in an active form (U.S. Patent Application Publication No. US/2006/0246539 A1 to Weiner et al.; Zhang et al., 2006). In Zhang et al., the E. coli expression vector pMS119 was used to construct the plasmid, pYebFH₆/MS, which expresses wild-type YebF protein under the control of an IPTG-inducible promoter and with a C-terminal hexa-His affinity tag. Analysis of the subcellular localization of the YebFH₆ protein after induction showed that the protein accumulated in the medium. To demonstrate that YebF could facilitate the export of other proteins, C-terminal fusions were made by inserting the coding sequences for mature alkaline phosphatase (E. coli phoA), α-amylase (Bacillus subtilis X-23, amy) and the human IL-2, between the C-terminal residue of YebF and the His tag. After induction, all three proteins were found to accumulate in the medium, indicating that the YebF protein could effect extracellular transport of the fused protein. Importantly, cytoplasmic proteins did not leak into the medium. YebF thus represents a potentially useful tool for facilitating the extracellular export of recombinant proteins.

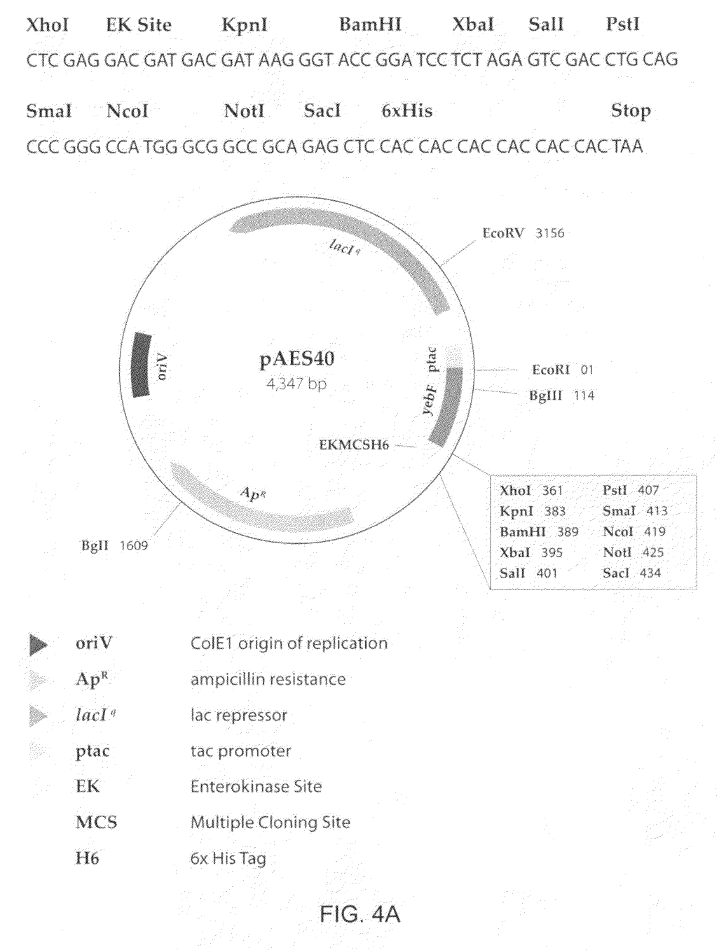

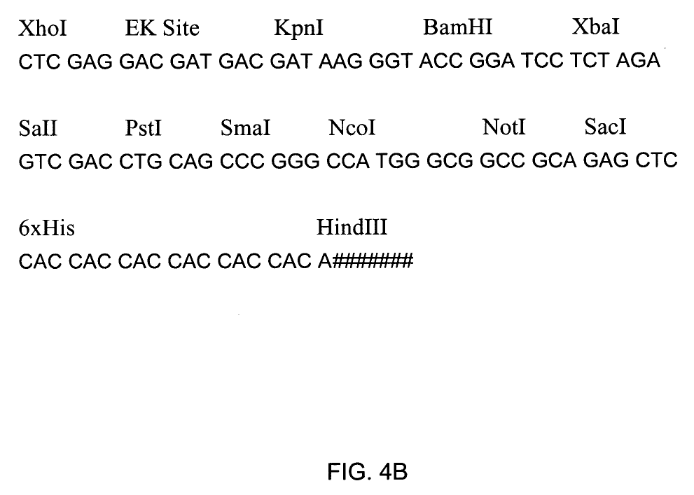

In one embodiment, the invention provides an expression vector comprising the plasmid map shown in FIGS. 4A and 4B. The expression vector, pAES40, facilitates the use of YebF as a carrier protein for the extracellular production of a recombinant protein, polypeptide or peptide. pAES40 carries the yebF nucleotide sequence, variant or portion thereof, which encodes the YebF protein or a biologically active variant or portion thereof. pAES40 was constructed by replacing the sequences extending from the YhoI to the HindIII sites of pYebFH₆/MS (Zhang et al., 2006) with the sequences shown in FIG. 4B. The yebF nucleotide sequence; origin of replication (oriV) from ColE1, a plasmid which is one of the most commonly used E. coli cloning vectors; the Ap^{R} gene (ampicillin-resistance); the lacI gene (lactose repressor); a promoter under the control of the lac operator/repressor system; a translation start site and a MCS were coupled together using standard techniques known in the art. The C-terminal amino acids of YebF are encoded by the sequence “CTC GAG” (SEQ ID NO: 16), with the remainder of the sequence depicting the reading frame of YebF (FIG. 4B). An enterokinase proteolytic cleavage site “GAC GAT GAC GAT AAG” (SEQ ID NO: 17) was placed between the MCS and the end of YebF to permit removal of the YebF sequences after export. A hexa-His sequence was placed at the end of the MCS to provide a His6 tag if needed; for example, for purification from the medium by affinity chromatography, or for identification with an antibody. The fusion of a target protein, polypeptide or peptide linked to the carboxyl end of YebF which is transported to growth medium thus facilitates the extracellular export of the desired protein, polypeptide or peptide, as described in Example 2. The use of YebF as a carrier for recombinant proteins provides a tool to circumvent toxicity and other contamination issues associated with protein production in E. coli.

Following assembly of any of the above expression vectors of the present invention, various techniques are available for introducing the expression vector into an appropriate host cell for expression of the recombinant protein, polypeptide or peptide. A “host cell” refers to a cell, irrespective, of the type, which expresses a nucleotide sequence encoding the protein, polypeptide or peptide within any of the expression vectors of the present invention and secretes the protein, polypeptide or peptide into the periplasm or extracellular medium. In one embodiment, the host cell is a prokaryote. In one embodiment, the host cell is E. coli. It will be appreciated by those skilled in the art that specific mutant strains of E. coli may permit higher levels of protein export. In one embodiment, the host cell is E. coli strain HB101, DH5α, JM109, HMS174, BLR or TOP10.

Non-limiting examples of techniques for introducing the expression vector into the host cell include electroporation, microinjection, liposome fusion, lipofection, lipopolyamine-mediated transfection, calcium-phosphate-DNA co-precipitation, biolistics, particle bombardment, polybrene-mediated transfection and other suitable techniques. The expression vector may become integrated into the genome of the host cell into which it is introduced, or may be present as unintegrated vector. Host cells carrying the expression vector are identified through the use of the selectable marker, and the presence of the gene of interest is confirmed by hybridization, PCR, antibodies, or other techniques.

The host cells are grown in growth medium until such time as is desired to harvest the secreted protein, polypeptide or peptide. The time required depends upon a number of factors relating to the bacterial expression system being used and to the target protein, polypeptide or peptide being produced. The rate of growth of a particular bacterial strain or species; the rate at which the secreted target protein, polypeptide or peptide accumulates in the periplasm or extracellular medium; the stability of the secreted target protein, polypeptide or peptide; and the time at which bacterial lysis begins to occur (which will contaminate the medium) are non-limiting examples of the types of considerations that will affect when the secreted target protein, polypeptide or peptide is harvested from the periplasm or extracellular medium.

In the case of intracellular production, the cells are harvested and the protein, polypeptide or peptide is released from the periplasm into the extracellular medium by inducing outer membrane leakage or rupturing the cells using mechanical forces, ultrasound, enzymes, chemicals and/or high pressure. Following secretion into the medium (for example, via YebF), the protein, polypeptide or peptide may be extracted from the medium. Depending upon the level of purity required, which will again depend upon the application for which the secreted recombinant protein, polypeptide or peptide will be used, the secreted protein may be further purified, for example by chromatography (e.g., affinity chromatography), precipitation, ultrafiltration, electrophoresis, or other suitable techniques.

The present invention provides significant advantages over current techniques of the prior art. Since the invention, in one embodiment, incorporates use of exported proteins, there is a significantly lower level of contamination, endotoxin, host cell proteins and nucleic acids, making purification easier and thus lowering production cost and durations. Importantly, the invention enables the production of proteins which might otherwise not be expressed due to toxicity and folding errors. The invention may be used for rapid production of proteins at a commercial scale, adapted to high throughput protein production, or readily employed in automated systems.

In one embodiment, the invention comprises a method of optimizing protein secretion. This is accomplished by expressing the protein of interest using a set of expression vectors that are designed to direct the export of the target protein to the periplasm or extracellular matrix using each of the three main protein secretion pathways of E. coli. The construct that secretes the most target protein identifies the optimal export pathway.

The Examples provided below are not intended to be limited to these examples alone, but are intended only to illustrate and describe the invention rather than limit the claims that follow.

### Example 1

### Secretion of Target Proteins into the Periplasm

To demonstrate that different proteins are exported differentially, the genes encoding alkaline phosphatase (PhoA) from E. coli and a streptavidin-luciferase hybrid protein (SA-Luc) were inserted downstream of the signal sequences. Each of these proteins is a marker for secretion into the periplasm. PhoA is only enzymatically active when exported to the periplasm (Manoil et al., 1990), and is non-functional if it accumulates in the cytoplasm. When SA-Luc (a heterologous semi-synthetic protein) is not exported, the protein forms inclusion bodies and does not exhibit biotin binding (streptavidin portion) or luminescent activity (luciferase portion).

Each of the expression vectors (carrying PhoA and SA-Luc fused to each of the signal sequences) was introduced into E. coli strain DH5α (phoA mutant). Expression of each protein was measured. For PhoA, the cells were cultured on solid medium containing isopropyl-thiogalactopyranoside (IPTG) to induce expression, and 5-bromo-4-chloro-3-indol-phosphate (a substrate of PhoA) to measure enzyme activity and thus indicate a Pho⁺ phenotype. When the PhoA protein was fused to the phoA (Sec), sufI (TAT) and torA (TAT) signal sequences, Pho⁺ cells were observed with the phoA signal sequence construct, in contrast to a marginal positive signal for the sufI and torA signals (data not shown). The ompA (Sec), dsbA (SRP) and torT (SRP) signal sequences did not yield Pho⁺ cells, indicating a lack of export. For PhoA, only the native signal sequence (Sec pathway) appeared to yield a high level of protein export.

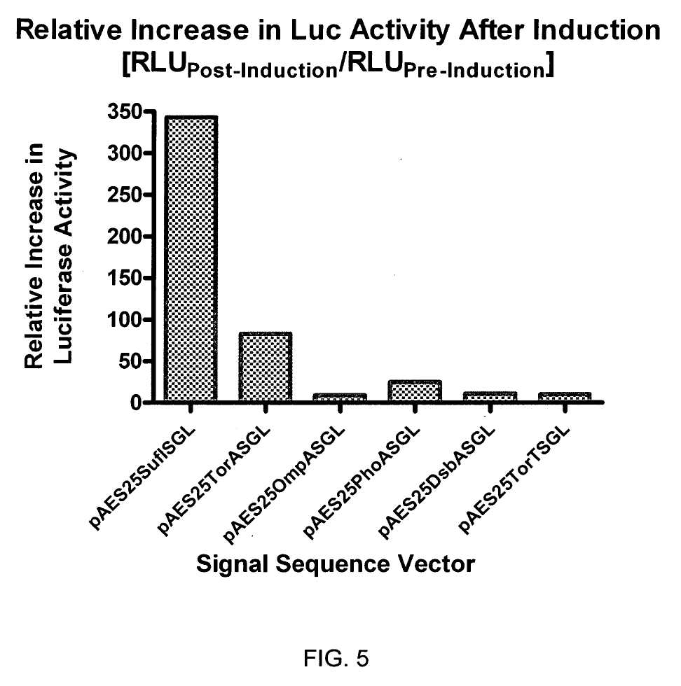

For SA-Luc, cultures were grown from single colonies to late exponential phase, and expression was induced by the addition of IPTG. After three hours post-induction, the cells were harvested and the enzyme activity was determined for pre- and post-induction samples. FIG. 5 shows the relative increase in luciferase activity after induction. Luciferase activity was significantly higher when either the sufI (TAT) and TorA (TAT) were used, but significantly lower when the SEC or SRP signal sequences were used. Little or no luciferase was produced after induction when the signal sequences for the SEC and SRP pathways were fused to SA-Luc. These experiments demonstrated that the type of signal sequence used and the protein export pathway to which the recombinant protein is directed have a profound effect on the level of target protein which accumulates.

### Example 2

### Recombinant Protein Production Utilizing YebF

The YebF export function works in several commonly used strains of E. coli for the expression of heterologous proteins including HB101, HMS174, BLR and TOP10. The plasmids pYebF-AmyH₆/MS (“YebF-Amy”) and pYebF-PhoAH₆/T7 (“YebF-PhoA”) were constructed according to U.S. Patent Application Publication No. US 2006/0246539 A1 to Weiner et al. (the contents of which are incorporated herein by reference in its entirety) E. coli strains carrying these plasmids were cultured from single colonies in 2 ml Terrific Broth medium (Tartof and Hobbs, 1987) supplemented with 100 μg/ml ampicillin overnight at 30° C. The overnight cultures were subcultured into 50 ml of fresh medium and incubated at 30° C. until the OD600 reached ˜0.6. A 6 ml sample was removed and IPTG was added to the remainder of the culture to a final concentration of 0.05 mM. The incubation continued for 22 hours. Samples were removed at 3, 8 and 22 hours post-induction and treated as follows: a 1 ml sample was microfuged for 2 min., the culture supernatant reserved and the cells suspended in water to give 10 OD per ml. The remaining 5 ml sample was centrifuged to separate the cells from the medium. The periplasmic and cytoplasmic fractions were prepared by cold osmotic shock and lysozyme/freeze-thaw, respectively. The corresponding fractions from the parent strains, MS119 (HB101/pMS119) and YebF (HB101/pYebFH6) were prepared similarly. Proteins which accumulated in whole cell extracts and the culture supernatant were analyzed by immunoblot and enzyme assay. For the immunoblot, equal volumes of medium or whole cell extracts prepared in SDS-PAGE loading dye were loaded onto 4-20% acrylamide gradient gels. The separated proteins were electroblotted to nitrocellulose. The His-tagged proteins were detected using monoclonal anti-His tag antibody. The enzyme assays were performed using cell-free extracts and culture supernatants as described in Zhang et al. (2006).

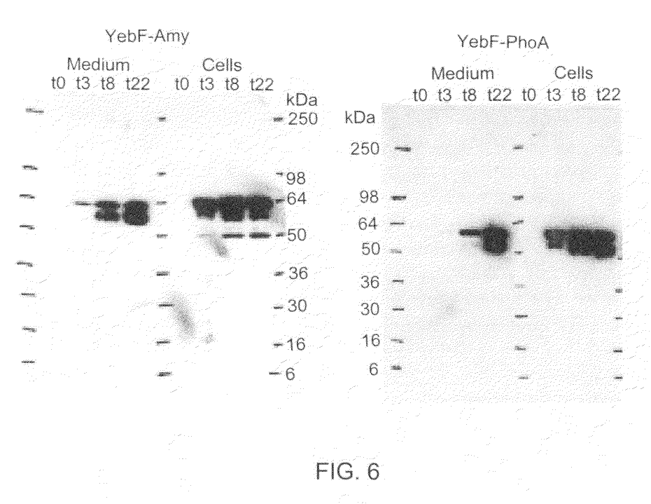

Both fusion proteins, YebF-Amy and YebF-PhoA, appeared in the bacterial growth medium and intracellularly following induction (FIG. 6). The proteins exhibited a time-dependent increase in export level following induction with IPTG. Their appearance within the cells preceded their accumulation in the medium, suggesting a rate-limiting process. The increase in enzyme activity for both fusion proteins paralleled the immunoblot (data not shown). The immunoblot showed that the proteins may have undergone a processing event beyond the expected removal of the signaling peptide such that the amino-terminal portion of each YebF was removed (the antibody used was an anti-His tag which is a C-terminal epitope). The basis processing is unknown, but peptide sequence analysis of the purified proteins revealed the resulting N-termini to be identical.

Accumulation of the fusion proteins in shake flask cultures was 20-50 mg/L, suggesting that with a fully optimized fermentation process, production levels could reach well over 100 mg/L. Further, strains harboring expression vectors which produce YebF-Amy and YebF-PhoA exhibited a growth impaired phenotype when cultured on medium containing 1 mM but not at 50 μM IPTG. In contrast, a strain expressing a YebF-GFP fusion was fully inhibited by 50 μM IPTG. In addition, the coding sequences of human GM-CSF and γ-interferon were subcloned into pYebFH₆/MS and showed that the GM-CSF protein was exported but that the interferon protein was not. As with the Amy and PhoA fusions, the GM-CSF construct exhibited a growth impaired phenotype when the cultures were induced with IPTG. These data taken with the observation of residual target protein in the cell lysates suggest a limitation which prevents full translocation of proteins that are over-expressed.

To determine if the export block of heterologous proteins fused to YebF can be alleviated using alternative export pathways, a set of vectors was constructed where the wild-type signal sequence of YebF was replaced with alternative signal sequences. The nucleotide sequence encoding the full-length YebF protein was subcloned into the MCS at the BamHI (5′ end) and SacI (3′ end) restriction sites of pAES25 to form “pAES25-YebF.” The nucleotide sequence encoding the mature YebF protein was subcloned into the MCS at the SacI (5′ end) and KpnI (3′ end) restriction sites of the pAES30 vector (to form “pAES25YebF_{DsbA}”) and pAES31 vector (to form “pAES25-YebF_{OmpA}”). Each plasmid was introduced into E. coli strain TOP10. The resulting strains were analyzed for their ability to express and export YebF protein to the bacterial growth medium. The experiments were performed in 25 ml shake flask cultures at 30° C. as described above. After 22 hours post-induction, the accumulation of YebF in the bacterial growth medium was determined by SDS-PAGE and immunoblot analyses.

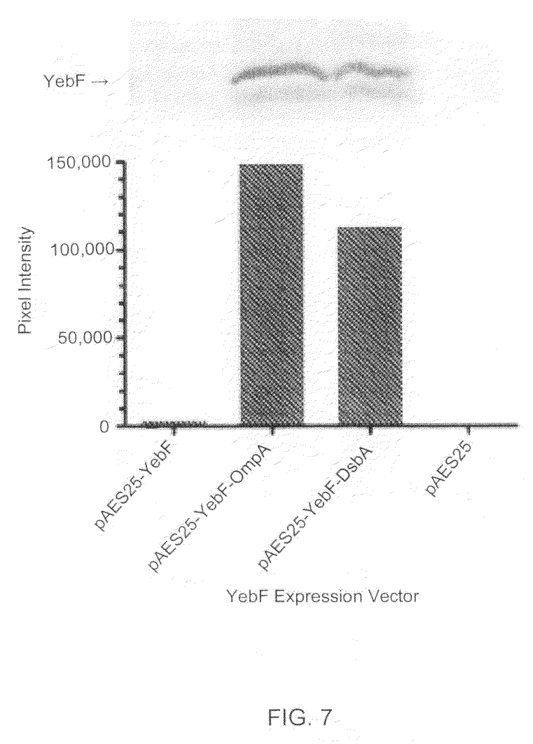

FIG. 7 shows the relative level of YebF accumulation in the medium. A portion of the stained gel corresponding to the location of YebF is shown above the graph. The x-axis labels correlate to the respective gel lane. The relative level of YebF accumulation was determined from the scanned gel image using TotalLab v2003.02 imaging software (Nonlinear Dynamics Ltd., UK). By exchanging the wild-type signal sequence of YebF for SEC (ompA)- or SRP (dsbA)-directed signal sequences, the level of YebF accumulation was increased by 46- and 35-fold, respectively. These data suggest that not only is YebF suitable for directing the translocation of recombinant proteins to the bacterial growth medium, but that by applying alternative signal sequences (which direct the proteins to the different export pathways), a significant increase in protein accumulation can be achieved.

### Example 3

### Identification of Mutations Affecting YebF Export

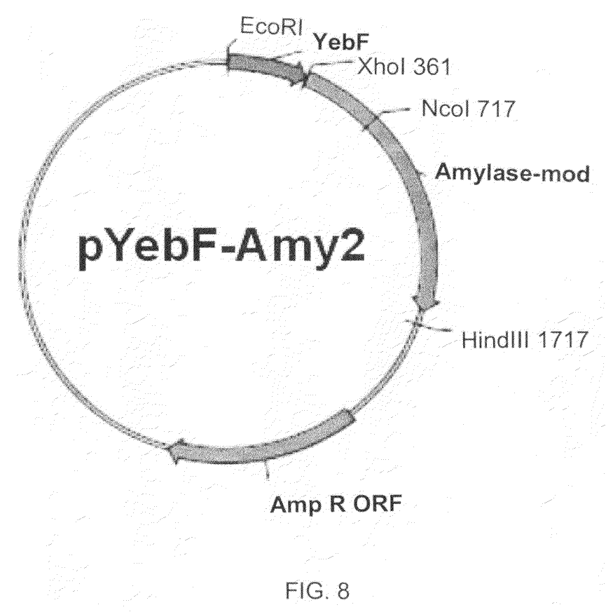

In order to identify mutations affecting YebF export, a simple genetic screen was devised. The screen uses medium containing Azure-starch to determine the amylose degrading phenotype of colonies. Starch degradation is indicated by the formation of a clear zone around the Amy-positive colonies. Amylase-negative colonies do not form clear zones and the medium remains uniformly blue in color. Wild-type E. coli is Amy-negative, whereas strains expressing the YebF-Amy fusion are Amy-positive. To develop the strain needed to screen for cis- and trans-acting mutations of YebF export function, a plasmid carrying a YebF-Amy fusion was prepared. This construct, designated pYebF-Amy2 (FIG. 8), differs from the original YebF-Amy fusion in that the wild-type yebF gene can be removed and replaced with DNA fragments containing mutations in the yebF coding sequence while retaining the integrity of the YebF-Amy fusion. This strain, when cultured on medium containing Azure-starch in the presence of 50 μM IPTG, forms clear zones around the colony whereas the parent strain harboring pYebFH₆ does not. Moreover, the strain harboring pYebF-Amy2 is able to grow on medium with 1% starch as the sole carbon source in contrast to the parent strain which can not. This screen is the basis for selecting cis- and trans-acting mutants.

Mutations were introduced into the wild-type yebF coding sequence using error-prone PCR. The resulting amplification products were subcloned into pYebF-Amy2 (FIG. 8) by exchanging the wild-type yebF for mutant yebF. The library of constructs was introduced into E. coli strain DH5α and the transformants scored on Azure-starch containing solid medium. Three mutant phenotypes were identified, (1) clear zones with a reduced area relative to the parent plasmid, (2) no clear zones, and (3) clear zones with an increased area (Table 4). DNA sequence analysis of the yebF portion of the plasmid purified from isolates representing each of the three phenotypes revealed the following: Strains showing no clear zone carried termination codons in the yebF sequence. Strains exhibiting a reduced or increase clear zones were amino acid substitutions at various locations within the yebF coding sequence.

TABLE 4
Phenotype and corresponding mutations in YebF mutants.

| Clone No. | Phenotype | YebF Sequence Mutations |
|---|---|---|
| 2 | No Halo | K34term; Q78P; V93A; G111term |
| 3 | Halo | G67S; V89I |
| 5 | Halo | C108S |
| 7 | Halo | I80T |
| 8 | No Halo | M1L(no start codon); S46I; ins A |
| 9 | Halo | G111R |
| 21 | No Halo | K64term; del T |
| 22 | Halo | K34E; E114G |
| 28 | Halo | L8S |
| 31 | Large Halo | N24K |
| 32 | No Halo | S27G, W75term |
| 34 | Small Halo | Q82K, S89term |
| 37 | Small Halo | D62V, D84V, I100T |

### REFERENCES

Baneyx, F. and Mujacic, M. (2004) Recombinant protein folding and misfolding in Escherichia coli. Nat. Biotech. 22:1399-1408.
Brinkmann, U., Chowdhury, P. S., Roscoe, D. M. and Pastan, I. (1995) Phage display of disulfide-stabilizing Fv fragments. J. Immunol. Methods 182:41-50.
Chang, C. N., Gray, G. L., Heyneker, H. L., and Rey, M. W. Secretion of heterologous proteins. U.S. Pat. No. 4,963,495, issued Oct. 16, 1990.
Choi, J. H. and Lee, S. Y. (2004) Export and extracellular production of recombinant proteins using Escherichia coli. Appl. Microbiol. Biotechnol. 64:625-635.
Cornelis, P. (2000) Expressing genes in different Escherichia coli compartments. Curr. Opin. Biotechnol. 11:450-454.
Fekkes, P. and Driessen, A. J. (1999) Protein targeting to the bacterial cytoplasmic membrane. Microbiol. Mol. Biol. Rev. 63:161-173.
Fisher, A. C. and DeLisa, M. P. A. (2004) A little help from my friends: quality control of preexport protein in bacteria. J. Bacteriol. 186:7467-7473.
Jung, S, and Pluckthun, A. (1997) Improving in vivo folding and stability of a single-chain Fv antibody fragment by loop grafting. Protein Eng. 10:959-966.
Koch, H. G., Moser, M. and Muller, M. (2003) Signal recognition particle-dependent protein targeting, universal to all kingdoms of life. Rev. Physiol. Biochem. Pharmacol. 146:55-94.
Krebber, A. Burmester, J. and Pluckthun, A. (1996) Inclusion of an upstream transcriptional terminator in phage display vectors abolishes background expression of toxic fusions with coat protein g3p. Gene 178:71-74.
Luirink, J. and Sinning, I. (2004) SRP-mediated protein targeting: structure and function revisited. Biochim. Biophys. Acta. 1694:17-35.
Manoil, C. Mekalanos, J. J. and Beckwith, J. (1990) Alkaline phosphatase fusions: Sensors of subcellular location. J. Bacteriol. 172:515-518.
Petro, J., Jackson, J. and Putney, S, Novel prokaryotic expression and Secretion system. U.S. Pat. No. 5,047,334, issued Sep. 10, 1991.
Rodi, D. J., Soares, A. S, and Makowski, L. (2002) Quantitative assessment of peptide sequence diversity in M13 combinatorial peptide phage display libraries. J. Mol. Biol. 322:1039-1052.
Sargent, F., Berks, B. C., and Palmer, T. (2005) Pathfinders and trailblazers: a prokaryotic targeting system for transport of folded proteins. FEMS Microbiol. Lett. 254:198-207.
Valent, Q. A. (2001) Signal recognition particle mediated protein targeting in Escherichia coli. Antonie Van Leeuwenhoek 79:17-31.
Weiner, J. and Zhang, G. Protein production method utilizing YebF. U.S. Patent Application Publication No. US 2006/0246539 A1, published Nov. 2, 2006.
Wulfing, C. and Pluckthun, A. (1993) Protein folding in the periplasm of Escherichia coli. Mol. Microbiol. 12(5):685-692.
Zhang, G., Brokx, S, and Weiner, J. H. (2006) Extracellular accumulation of recombinant proteins fused to the carrier protein YebF in Escherichia coli. Nat. Biochem. 24:100-104.

All publications mentioned in this specification are indicative of the level of skill of those skilled in the art to which this invention pertains. All publications are herein incorporated by reference to the same extent as if each individual publication was specifically and individually indicated to be incorporated by reference.

What is claimed is:

1. An expression vector capable of directing the expression and secretion of a protein, polypeptide or peptide in a suitable host cell, wherein the expression vector comprises a nucleic acid encoding a fusion protein comprising yebF, or a biologically active variant or portion thereof, and the protein, polypeptide or peptide, operably linked to control sequences compatible with the host cell, and a secretory signal sequence for directing the secretion of the fusion protein.

2. The expression vector of claim 1, wherein the signal sequence comprises one of SEQ ID NO: 4, SEQ ID NO: 5, SEQ ID NO: 6, SEQ ID NO: 7, SEQ ID NO: 8 or SEQ ID NO: 9.

3. The expression vector of claim 1, wherein the expression vector is comprised of one of plasmids pAES30, pAES31, pAES32, pAES33, pAES34, pAES35, or pAES40.

4. The expression vector of claim 1, for use in a host cell selected from a prokaryote, Escherichia coli or a strain thereof.

5. An isolated host cell transformed by the expression vector of claim 1, wherein said cell expresses and secretes the fusion protein.

6. The isolated host cell of claim 5, wherein the host cell is selected from a prokaryote, Escherichia coli or a strain thereof.

7. A method of optimized production of a protein, polypeptide or peptide comprising the steps of:
(a) choosing an expression vector as claimed in claim 1, comprising a signal sequence associated with one of SEC, TAT, or SRP export pathway, wherein said choice is made having regard to known information about the protein, polypeptide or peptide, or experimental information from expression studies of the signal sequence and the protein, polypeptide or peptide;
(b) transforming a suitable host cell with the chosen expression vector;
(c) culturing the transformed host cell under conditions conducive to the expression of the protein, polypeptide or peptide to generate a secreted protein, polypeptide or peptide; and
(d) recovering the secreted protein, polypeptide, or peptide from the host cell, from the culture medium comprising the host cell, or from an extract obtained from the host cell.

8. The method of claim 7 wherein expression vector comprises one of plasmids pAES30, pAES31, pAES32, pAES33, pAES34, pAES35 or pAES40.

9. The method of claim 7, wherein the expression vector comprises a signal sequence comprises one of SEQ ID NO: 4, SEQ ID NO: 5, SEQ ID NO: 6, SEQ ID NO: 7, SEQ ID NO: 8 or SEQ ID NO: 9.

10. The method of claim 10, further comprising the step of purifying the secreted fusion protein and isolating the protein, polypeptide or peptide.

11. The method of claim 8, wherein the host cell is Escherichia coli or a strain thereof.


SEQUENCE LISTING (18 sequences)

SEQ ID NO: 1 - protein, 118 aa, Escherichia coli
MKKRGAFLGLLLVSACASVFAANNETSKSVTFPKCEDLDAAGIAASVKRDYQQNRVARWADDQKIVGQADPVAWVSLQDIQGKDDKWSVPLTVRGKSADIHYQVSVDCKAGMAEYQRR

SEQ ID NO: 2 - protein, 97 aa, Escherichia coli
ANNETSKSVTFPKCEDLDAAGIAASVKRDYQQNRVARWADDQKIVGQADPVAWVSLQDIQGKDDKWSVPLTVRGKSADIHYQVSVDCKAGMAEYQRR

SEQ ID NO: 3 - DNA, 357 bp, Escherichia coli
atgaaaaaaagaggggcgtttttagggctgttgttggtttctgcctgcgcatcagttttcgctgccaataatgaaaccagcaagtcggtcactttcccaaagtgtgaagatctggatgctgccggaattgccgcgagcgtaaaacgtgattatcaacaaaatcgcgtggcgcgttgggcagatgatcaaaaaattgtcggtcaggccgatcccgtggcttgggtcagtttgcaggacattcagggtaaagatgataaatggtcagtaccgctaaccgtgcgtggtaaaagtgccgatattcattaccaggtcagcgtggactgcaaagcgggaatggcggaatatcagcggcgttaa

SEQ ID NO: 4 - DNA, 60 bp, Artificial
  Synthetic DNA based on sequence from Escherichia coli gene; Encodes signal sequence (PhoA)
aaacaaagcactattgcactggcactcttaccgttactgtttacccctgtgacaaaagcg

SEQ ID NO: 5 - DNA, 60 bp, Artificial
  Synthetic DNA based on sequence from Escherichia coli gene; Encodes signal sequence (OmpA)
aaaaagacagctatcgcgattgcagtggcactggctggtttcgctaccgtagcgcaggcg

SEQ ID NO: 6 - DNA, 54 bp, Artificial
  Synthetic DNA based on sequence from Escherichia coli gene; Encodes signal sequence (DsbA)
aaaaagatttggctggcgctggctggtttagttttagcgtttagcgcatcggcg

SEQ ID NO: 7 - DNA, 51 bp, Artificial
  Synthetic DNA based on sequence from Escherichia coli gene; Encodes signal sequence (TorT)
cgcgtactgctatttttacttctttcccttttcatgttgccggcattttcg

SEQ ID NO: 8 - DNA, 92 bp, Artificial
  Synthetic DNA based on sequence from Escherichia coli gene; Encodes signal sequence (SufI)
tcactcagtcggcgtcagttcattcaggcatcggggattgcactttgtgcaggcgctgttccactgaaggccagcgcagcagatctactagt

SEQ ID NO: 9 - DNA, 128 bp, Artificial
  Synthetic DNA based on sequence from Escherichia coli gene; Encodes signal sequence (TorA)
aacaataacgatctctttcaggcatcacgtcggcgttttctggcacaactcggcggcttaaccgtcgccggtatgctgggtccgtcattgttaacgccgcgacgtgcgacggcagcagatctactagt

SEQ ID NO: 10 - protein, 21 aa, Artificial
  Signal sequence (PhoA) encoded by SEQ ID NO4
MKQSTIALALLPLLFTPVKTA

SEQ ID NO: 11 - protein, 21 aa, Artificial
  Signal sequence (OmpA) encoded by SEQ ID NO5
MKKTAIAIAVALAGFATVAQA

SEQ ID NO: 12 - protein, 19 aa, Artificial
  Signal sequence (DsbA) encoded by SEQ ID NO6
MKKIWLALAGLVLAFSASA

SEQ ID NO: 13 - protein, 17 aa, Artificial
  Signal sequence (TorT) encoded by SEQ ID NO7
MRVLLFLLSLFMLPAFS

SEQ ID NO: 14 - protein, 27 aa, Artificial
  Signal sequence (SufI) encoded by SEQ ID NO8
MSLSRRQFIQASGIALCAGAVPLKASA

SEQ ID NO: 15 - protein, 39 aa, Artificial
  Signal sequence (TorA) encoded by SEQ ID NO9
MNNNDLFQASRRRFLAQLGGLTVAGMLGPSLLTPRRATA

SEQ ID NO: 16 - DNA, 6 bp, Artificial
  Synthetic DNA encoding C-terminal amino acids of YebF; Restriction site
ctcgag

SEQ ID NO: 17 - DNA, 15 bp, Artificial
  Synthetic DNA; consensus sequence for enterokinase proteolytic cleavage site
gacgatgacgataag

SEQ ID NO: 18 - DNA, 4661 bp, Artificial
  Vector pAES25 derived from Escherichia coli and synthetic DNAs
ctcgagaaatcataaaaaatttatttgctttgtgagcggataacaattataatagattcaattgtgagcggataacaatttcacacagaattcattaaagaggagaaattaactatgggatccgcatgcgagctcggtaccccgggtcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccctctagattacgtgcagtcgatgataagctgtcaaacatgagaattgtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcggaatttcgggcagcgttgggtcctggccacgggtgcgcatgatctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccatgacattaacctataaaaataggcgtatcacgaggccctttcgtcttcac